(19) United States
(12) Patent Application Publication
(10) Pub. No.: US 2012/0071961 A1
(43) Pub. Date: Mar. 22, 2012

(54) Stent With Hollow Drug-Eluting Elements

(75) Inventor: Ryan Bienvenu, Santa Rosa, CA (US)

(73) Assignee: Medtronic Vascular, Inc., Santa Rosa, CA (US)

(21) Appl. No.: 12/884,287

(22) Filed: Sep. 17, 2010

(51) Int. Cl. A61F 2/84; B65B 1/04

(52) U.S. Cl. 623/1.16; 623/1.34; 623/1.42; 141/1

(57) ABSTRACT

A stent includes a plurality of cylindrical elements joined along a common longitudinal axis to form a tube. The cylindrical elements include struts joined by crowns. Hollow, drug-eluting elements are disposed between adjacent cylindrical elements and connect adjacent cylindrical elements to each other. A therapeutic substance fills the lumen of the drug-eluting elements, and openings in the walls of the drug-eluting elements allow elution of therapeutic substance from the lumen for treatment of a vessel.

### FIELD OF THE INVENTION

The present invention relates to implantable medical devices that release a therapeutic substance and methods of forming such medical devices.

### BACKGROUND OF THE INVENTION

Drug-eluting implantable medical devices have become popular in recent times for their ability to perform their primary function (such as structural support) and their ability to medically treat the area in which they are implanted.

For example, drug-eluting stents have been used to prevent restenosis in coronary arteries. Drug-eluting stents may administer therapeutic agents such as anti-inflammatory compounds that block local invasion/activation of monocytes, thus preventing the secretion of growth factors that may trigger VSMC proliferation and migration. Other potentially anti-restenotic compounds include antiproliferative agents, such as chemotherapeutics, which include rapamycin and paclitaxel. Other classes of drugs such as anti-thrombotics, anti-oxidants, platelet aggregation inhibitors and cytostatic agents have also been suggested for anti-restenotic use.

Drug-eluting medical stents may be coated with a polymeric material which, in turn, is impregnated with a drug or a combination of drugs. Once the stent is implanted at a target location, the drug is released from the polymer for treatment of the local tissues. The drug is released by a process of diffusion through the polymer layer for biostable polymers, and/or as the polymer material degrades for biodegradable polymers.

Controlling the rate of elution of a drug from the drug impregnated polymeric material is generally based on the properties of the polymer material. However, at the conclusion of the elution process, the remaining polymer material in some instances has been linked to an adverse reaction with the vessel, possibly causing a small but dangerous clot to form. Further, drug impregnated polymer coatings on exposed surfaces of medical devices may flake off or otherwise be damaged during delivery, thereby preventing the drug from reaching the target site. Still further, drug impregnated polymer coatings are limited in the quantity of the drug to be delivered by the amount of a drug that the polymer coating can carry and the size of the medical devices. Controlling the rate of elution using polymer coatings is also difficult.

Stents made from a hollow-tubular wire filled with therapeutic agents have been proposed. However, forming a hollow-wire stent by bending a hollow-wire into a stent form may cause kinking, cracking, or other undesirable properties in the finished stent. Accordingly, co-pending U.S. application Ser. No. 12/500,359, filed Jul. 9, 2009, incorporated by reference herein in its entirety, describes methods for forming a hollow-wire stent by forming a core wire, bending the core wire into the selected stent shape, and then removing the sacrificial or inner member of the core wire. Provisional application No. 61/244,049, filed Sep. 20, 2009, incorporated by reference herein in its entirety, describes additional methods for forming a hollow-wire stent. Further, filling a hollow-wire stent may be problematic due to the small size and tortuous bends in the stent structure. Provisional application No. 61/244,050, filed Sep. 20, 2009, incorporated by reference herein in its entirety, describes methods for filling a hollow-wire stent.

Accordingly, drug-eluting stents are needed that utilize the advantages of a hollow-wire stent, such as the ability to delivery increased quantities of the therapeutic substance and improved control of the elution rate of the therapeutic substance, while reducing potential manufacturing difficulties of a hollow-wire stent.

### BRIEF SUMMARY OF THE INVENTION

A stent includes a plurality of cylindrical elements joined along a common longitudinal axis to form a tube. The cylindrical elements include struts joined by crowns. Hollow, drug-eluting elements are disposed between adjacent cylindrical elements and connect adjacent cylindrical elements to each other. A therapeutic substance fills the lumen of the drug-eluting elements, and openings in the walls of the drug-eluting elements allow elution of therapeutic substance from the lumen for treatment of a vessel.

In a method of forming a stent, a plurality of cylindrical elements are formed, wherein each cylindrical element includes a plurality of struts connected together by a series of crowns. A plurality of hollow drug-eluting elements are formed, with the drug-eluting elements including at least one opening through a wall thereof. A lumen of the drug-eluting elements is filled with a therapeutic substance, and the drug-eluting elements are connected between the plurality of cylindrical elements such that ends of each drug-eluting element are connected to adjacent cylindrical elements such that the cylindrical elements are connected together and aligned generally along a common longitudinal axis to form the stent. The step of filling the drug-eluting elements with a therapeutic substance may take place before or after connecting the drug-eluting elements to the cylindrical elements.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a schematic illustration of an exemplary stent in accordance with an embodiment hereof.

FIG. 2 is a close-up view of a portion of the stent of FIG. 1.

FIG. 3 is a cross-section taken along line 3-3 of FIG. 2.

FIG. 4 is a cross-section taken along line 404 of FIG. 2.

FIG. 5 is a perspective view of a cylindrical element of the stent of FIG. 1.

FIG. 6 is a schematic illustration of a portion of a vessel with a stent disposed therein.

FIG. 7 is a longitudinal cross-section of an embodiment of drug-eluting element.

FIG. 8 is a perspective view of an embodiment of a drug-eluting element.

FIG. 9 is a longitudinal cross-section of an embodiment of a drug-eluting element.

FIG. 10 is a longitudinal cross-section of an embodiment of a drug-eluting element.

FIG. 11 is a longitudinal cross-section of an embodiment of a drug-eluting element.

FIGS. 12-18 show schematic illustrations of portions of stents including drug-eluting elements disposed between cylindrical elements.

FIG. 19 illustrates an embodiment of a method of forming a stent.

FIG. 20 illustrates an embodiment of a method of forming a stent.

FIG. 21 illustrates an embodiment of a method of filling a stent with a therapeutic substance.

### DETAILED DESCRIPTION OF THE INVENTION

Specific embodiments of the present invention are now described with reference to the figures, where like reference numbers indicate identical or functionally similar elements.

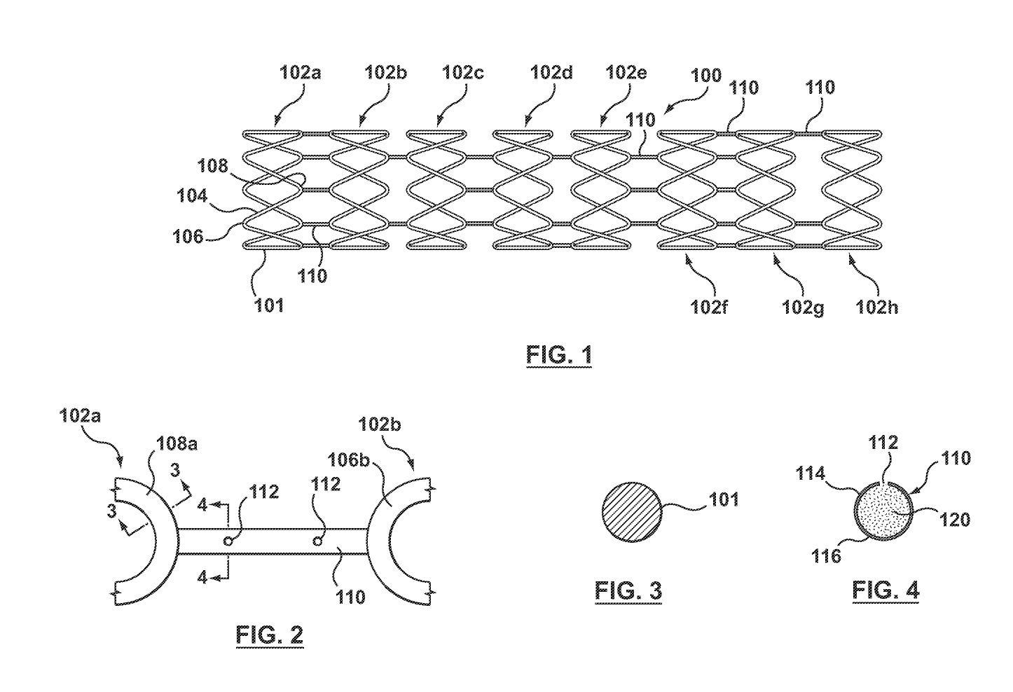

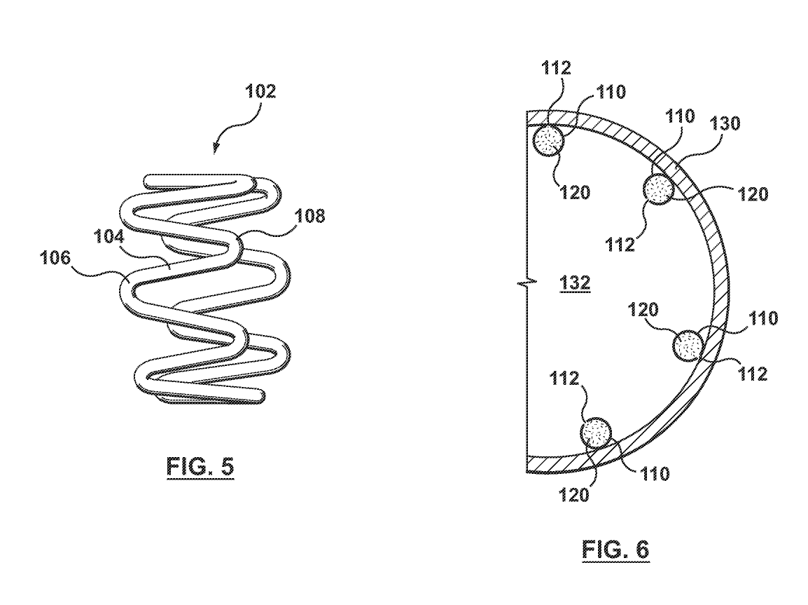

An embodiment of a stent 100 disclosed herein is shown in FIG. 1. In particular, stent 100 includes a plurality of cylindrical elements or bands 102 joined along a common longitudinal axis to form a tube. In the embodiment shown, there are eight cylindrical elements 102a-102h joined together to form stent 100, but as would be apparent to those of ordinary skill in the art, more or less cylindrical elements 102 can be used. Each cylindrical element 102 is formed from a wire 101 that is bent into a series of generally sinusoidal waves including generally straight segments or struts 104 joined by bent segments or crowns 106, 108. Crowns on the left side of each cylindrical element 102 have been labeled with reference numeral 106 and crowns on the right side of each cylindrical element 102 have been labeled with reference numeral 108. Those of ordinary skill in the art would recognize that such labeling is arbitrary and merely for the purpose of description herein. FIG. 5 shows a perspective view of a cylindrical element 102. Cylindrical elements 102 may be identical to or similar to cylindrical elements found in the Driver® coronary stent, currently available from the assignee of the present invention, Medtronic Vascular, Inc. The invention hereof is not limited to the pattern shown in FIG. 1. Drug-eluting elements 110 are disposed between adjacent cylindrical elements 110 and connect adjacent cylindrical elements 102 to each other, as shown in FIG. 1.

FIG. 2 illustrates a close-up of a crown 108a of a first cylindrical element 102a, a drug-eluting element 110, and a crown 106b of a second cylindrical element 102b adjacent the first cylindrical element. As shown in FIG. 2, drug-eluting element 110 includes at least one opening 112 for eluting a drug contained within drug-eluting element 110. As shown in the cross-sectional views of FIGS. 3 and 4, wire 101 of cylindrical elements 102 is a solid wire (FIG. 3) and drug-eluting element 110 is a hollow wire (FIG. 4). Those of ordinary skill in the art would understand that wire 101 need not be solid, however, wire 101 forming cylindrical elements 102 is not drug-filled. Wire 101 may be formed from, for example and not by way of limitation, stainless steel, cobalt-chromium alloys (such as F-562 cobalt-chromium alloy), cobalt-nickel-chromium-molybdenum alloys (such as MP35N and MP20N), nickel-titanium alloys (such as Nitinol), magnesium, cobalt-chromium-tungsten-nickel alloys (such as L605), combinations thereof, or other suitable materials known to those of ordinary skill in the art. Drug-eluting element 110 is a hollow tube having a relatively thin wall 114 and a lumen 116. Lumen 116 is filled with a therapeutic substance 120. Wall 114 of drug-eluting element 110 may be made of the materials listed above for wire 101, or any other suitable materials known to those of ordinary skill in the art.

Because drug-eluting elements 110 are not primarily responsible for radial support of stent 100, the wall of the drug-eluting element may be thinner than a hollow tube used for radial support. For example, and not by way of limitation, an outer diameter of drug-eluting element 100 may be in the range of 0.002 inch to 0.004 inch. The thickness of wall 114 may be in the range of 0.0005 inch to 0.001 inch. In an embodiment, struts 104 may be approximately 0.01 inch to 0.06 inch in length and drug-eluting elements 110 may also be approximately 0.01 inch to 0.06 inch in length. As shown in FIGS. 3 and 4, both wire 101 of cylindrical elements 102 and drug-eluting elements 110 are round in cross-section. However, those of ordinary skill in the art would recognize that other shapes are equally acceptable, such as oval, elliptical, half-round or D-shaped, rectangular, etc.

Drug-eluting elements 110 are connected to cylindrical elements 102 by fusion, welding, soldering, adhesive, or other mechanical or chemical connections known to those skilled in the art. As shown in FIG. 1, the quantity of drug-eluting elements 110 between adjacent cylindrical elements 102 may vary from stent to stent, or within a stent itself. For example, more drug-eluting elements 110 may be disposed between cylindrical elements 102 near the ends of stent 100 and less between the cylindrical elements 102 near the middle of stent 100, or vice versa. Such variation permits a variable drug-elution profile along the length of stent 100. Further, instead of or in addition to varying the amount of drug-eluting elements 110, the size, therapeutic substance, or size or quantity of openings 112 may be varied from stent to stent or within a stent. Different sized drug-eluting elements 110, in diameter or length, permit different amounts of a therapeutic substance 120 disposed within lumen 116. Different sized openings 112 permit variable elution rates, as larger holes generally provide faster elution. Similarly, different quantities of openings 112 in drug-eluting elements 110 provide variable elution rates and profiles. Openings 112 may be, for example and not by way of limitation, 10-30 μm in diameter.

Further, because the drug-eluting elements 110 are individual, different drug-eluting elements may include different therapeutic substances. Further, it would be understood by those of ordinary skill in the art that some elements between cylindrical elements 102 may be solid wire, for example and not by way of limitation, if such elements are not needed for drug-elution but may be needed for other reasons, such as scaffolding.

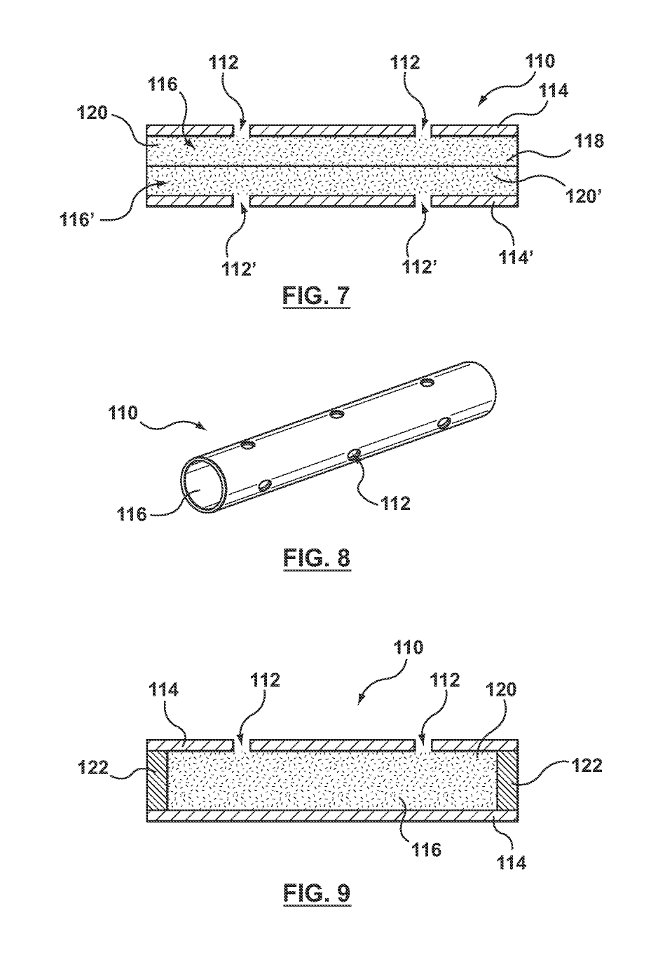

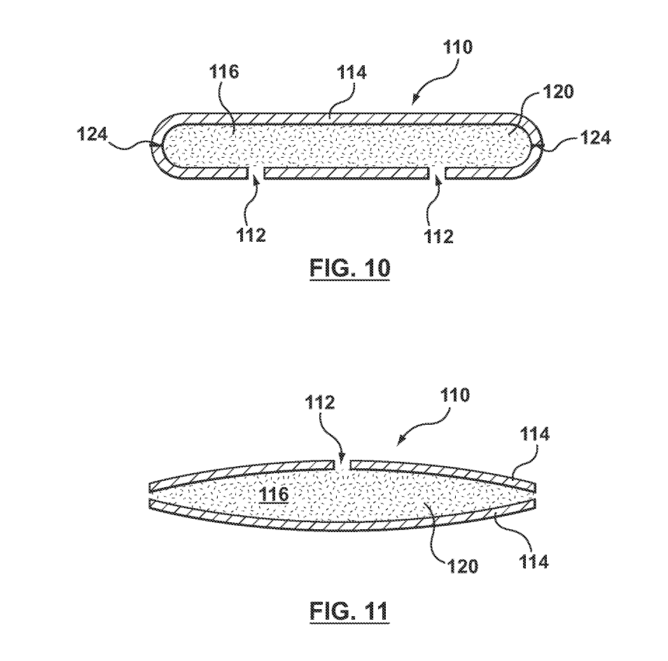

FIG. 6 illustrates schematically a cross-section of a portion of a stent 100 disposed in a vessel 130. In this example, some drug-eluting elements 110 between adjacent cylindrical elements 102 are shown. Further, openings 112 in alternating drug-eluting elements 110 face different sides of the stent. As shown in FIG. 6, starting from 12:00 on a clock and progressing clockwise, the first and third drug-eluting elements 110 have holes 112 facing outwardly or the abluminal side of the stent, while the second and fourth drug-eluting elements have openings 112 facing inwardly or the luminal side of the stent. Using such a configuration, a first therapeutic substance, such as an anti-proliferative drug, could be used to fill the drug-eluting elements 110 with outwardly facing openings 112, and a second therapeutic substance different than the first therapeutic substance, such as an anti-coagulant drug, could be used to fill the drug-eluting elements 110 having inwardly facing openings 112. It would be appreciated by those of ordinary skill in the art that the first therapeutic substance and the second therapeutic substance need not necessarily be different. Thus, the same drug may be eluted from drug-eluting elements 110 with outwardly facing openings 112 and drug-eluting elements with inwardly facing openings 112. As would be further understood by those of ordinary skill in the art, individual drug-eluting elements 110 can have elution openings 112 only on an outwardly facing or abluminal surface of stent 100 (as shown in FIG. 9), only on the inwardly facing or luminal surface of stent 100 (as shown in FIG. 10), both surfaces (as shown in FIG. 7), or may be provided anywhere along the circumference of the drug-eluting element 110 (as shown in FIG. 8).

In another embodiment shown in FIG. 7, a drug-eluting element 110 includes a divider or barrier 118 dividing lumen 116 into two sections. Barrier 118 extends longitudinally such that a lumen 116 is disposed on the abluminal side of drug-eluting element 110 and a second lumen 116′ is disposed on the lumen side of drug-eluting element 110. A first therapeutic substance 120 is disposed in lumen 116 and a second therapeutic substance 120′ is disposed in lumen 116′. Openings 112 on the abluminal or outwardly facing surface of drug-eluting element 110 allow first therapeutic substance 120 to elute from lumen 116 and openings 112′ on the luminal or inwardly facing surface allow second therapeutic substance 120′ to elute from lumen 116′. For example, and not by way of limitation, an anti-proliferative drug may be disposed in lumen 116 and an anti-coagulant drug may be disposed in lumen 116′. Those of ordinary skill in the art would recognize the benefits of other drug combinations in such an embodiment.

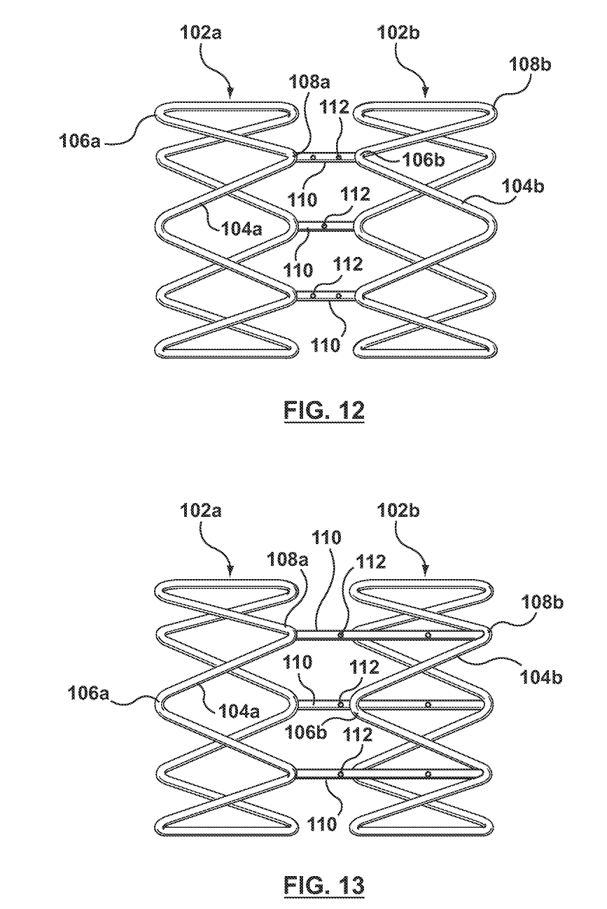

FIGS. 12-18 show various embodiments of how drug-eluting elements 110 may be placed between cylindrical elements 102 of a stent. In FIGS. 12-18, only two cylindrical elements 102a, 102b of a stent are shown for convenience, but one of ordinary skill in the art would recognize that the embodiments shown can be used between any two cylindrical elements 102 of a stent with any number of cylindrical elements, and that different embodiments can be used for the same stent and may be combined. Further, the portions of wire cylindrical elements 102 and drug-eluting elements 110 shown in dashed lines indicate that these portions are behind the solid lines as the stent is tubular.

In FIG. 12, the cylindrical elements 102a, 102b are offset such that crowns 108a of the first cylindrical element 102a align longitudinally with crowns 106b of the second cylindrical element 102b. The drug-eluting elements 110 are connected to crowns 108a of the first cylindrical element 102a and crowns 106b of the second cylindrical element 102b in what is generally referred to in the art as a peak-to-peak connection.

In FIG. 13, the cylindrical elements 102a, 102b are aligned such that crowns 108a of the first cylindrical element 102a align longitudinally with crowns 108b of the second cylindrical element 102b. The drug-eluting elements 110 are connected between crowns 108a of the first cylindrical elements 102a and crowns 108a of the second cylindrical element 102b in what is generally referred to in the art as a peak-to-valley connection.

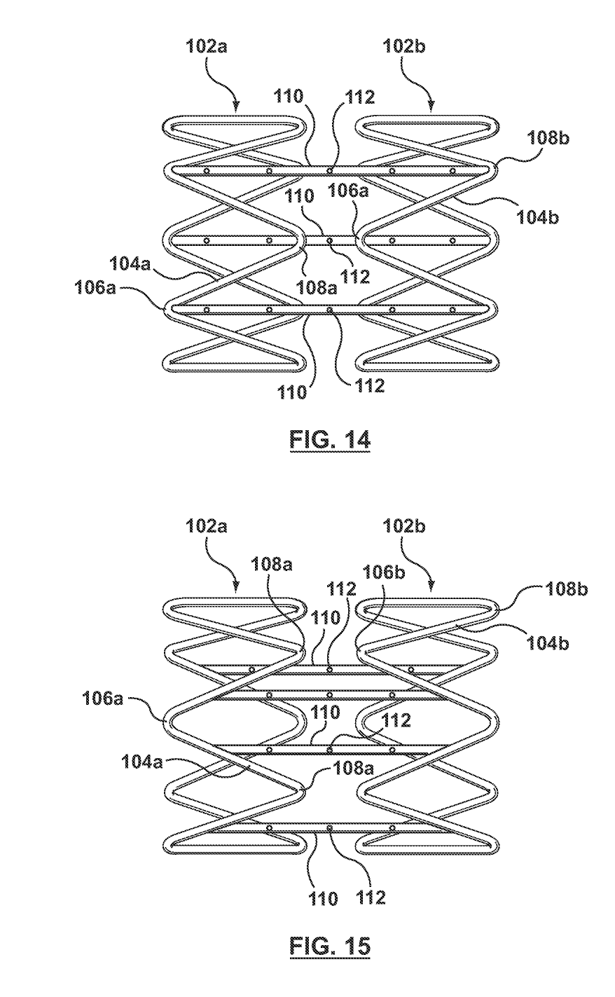

In FIG. 14, the cylindrical elements 102a, 102b are offset as in FIG. 12 such that crowns 108a of the first cylindrical element 102a align longitudinally with crowns 106b of the second cylindrical element 102b and crowns 106a of the first cylindrical element 102a align with crowns 108b of the second cylindrical element 102b. The drug-eluting elements 110 of the embodiment of FIG. 14, however, are connected between crowns 106a of the first cylindrical element 102a and crowns 108b of the second cylindrical element 102b in what is generally referred to in the art as a valley-to-valley connection.

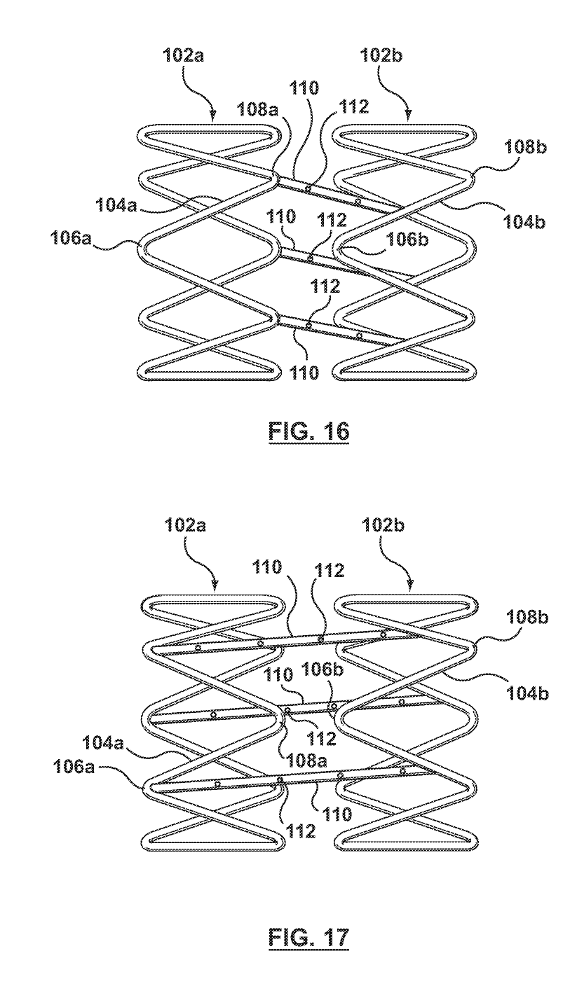

In FIG. 15, the drug-eluting elements 110 are connected between a strut 104a of the first cylindrical element 102a and a strut 104b of the second cylindrical element 102b in what is generally referred to in the art as a strut-to-strut connection. In FIG. 16 the drug-eluting elements 110 are connected between a crown 108a of the first cylindrical element 102a and a strut 104b of the second cylindrical element 102b in what can be referred to as a peak-to-strut connection. In FIG. 17 the drug-eluting elements 110 are connected between a crown 106a of the first cylindrical element 102a and a strut 104b of the second cylindrical element 102b in what can be referred to as a valley-to-strut connection.

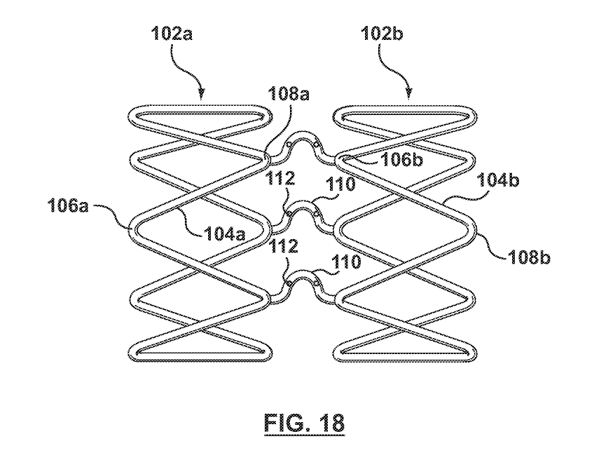

In FIG. 18, the cylindrical elements 102a, 102b are offset as in FIG. 12 such that crowns 108a of the first cylindrical element 102a align longitudinally with crowns 106b of the second cylindrical element 102b. The drug-eluting elements 110 in FIG. 18 are curved or bended and are connected to crowns 108a of the first cylindrical element 102a and crowns 106b of the second cylindrical element 102b in a peak-to-peak connection. The bends of the drug-eluting elements 110 of FIG. 18 are in the same directing such that the drug-eluting elements 110 may nest together when the stent is in a radially compressed configuration for delivery. However, those of ordinary skill in the art would recognize that the bends need not face the same direction. Further, those of ordinary skill in the art would recognize that curved drug-eluting elements may be used in each of the embodiments described in FIGS. 13-17.

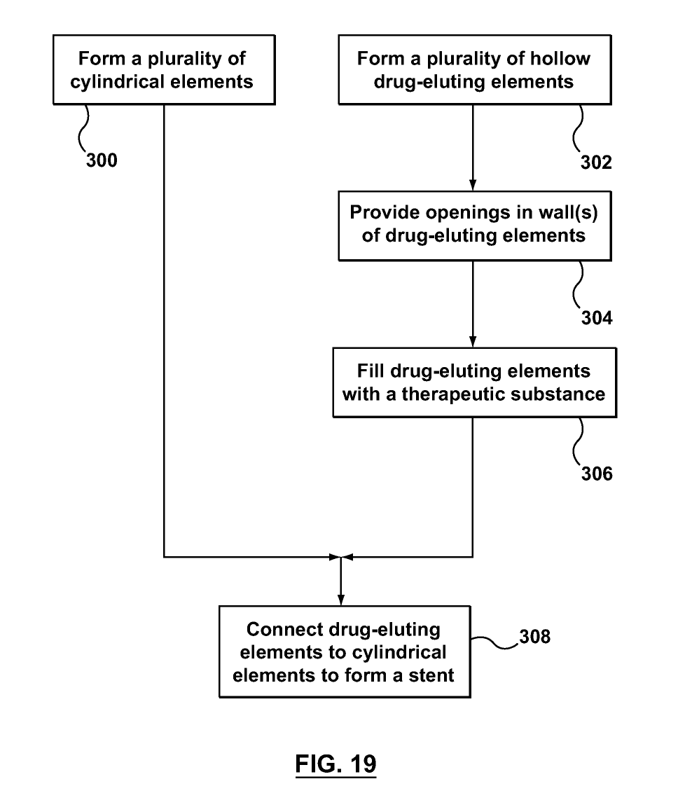

FIG. 19 outlines a method for forming a stent 100 in accordance with an embodiment hereof. In step 300, cylindrical elements 102 are formed. In step 302, drug-eluting elements 110 are formed without a therapeutic substance disposed therein. In step 304, openings 112 are formed in wall 114 of drug-eluting elements 110. Openings 112 may be laser cut, drilled, etched, or otherwise provided in wall 114 of drug-eluting element 110. In step 306, lumens 116 of drug-eluting elements 110 are filled with a therapeutic substance 120. Lumens 116 may be filled by any method known to those of ordinary skill in the art, for example: (1) solution fill via coupling to end of hollow element; (2) use of an azeotropic solution to evaporate mixture of solvents off, leaving solid drug behind (in this instance, hollow elements would be placed in a sonicating bath of this solvent mixture and drug); (3) a sonicating bath of solvent and drug in a cryochamber that allows for solvent to be sublimated, leaving dry drug behind, and methods described in U.S. Provisional Application No. 61/244,050, filed Sep. 20, 2009, which is incorporated by reference herein in its entirety. Further, because drug-eluting elements 110 are filled prior to attachment to cylindrical elements 102, the open ends of lumen 116 may assist in filling the drug-eluting element 110. However, the ends may be closed off, as described in more detail herein, and lumen 116 may be filled through openings 112 or other, larger openings made for filling lumen 116. These additional filling openings are closed prior to using the stent. After lumens 116 of drug-eluting elements 110 are filled, the drug-eluting elements 110 are connected to cylindrical elements 102 in step 308 to form a stent.

In the method described above, if drug-eluting elements 110 are fused or welded to cylindrical elements 102 and the therapeutic substance 120 is sensitive to heat, an insulative material or heat-sink may be disposed at each end of drug-eluting element 110. As shown in FIG. 9, plugs 122 may be provided at each end of drug-eluting element 110. Plugs 122 may be made from a relatively insulative material, such as a polymer or composite ceramic-polymer material. Examples include polyimide, PTFE, glass reinforced or impregnated PTFE and glass reinforced or impregnated polyimide. Such an insulative plug 122 insulates therapeutic substance 120 from heat as drug-eluting elements 110 are fused or welded to cylindrical elements 102. Plugs 122 may be connected to drug-eluting elements 110 by fusion, welding, adhesive, compression fit, or other connections known to those of ordinary skill in the art. Alternatively, plugs 122 may be a made of a conductive material whereby the generated heat is quickly dissipated into the surrounding stent or additional heat sink. Examples of conductive materials include cobalt based alloys, steels, gold, tantalum, platinum-iridium alloys and others. In some embodiments, plugs 122 may be made radiopaque by adding radiopaque material such as barium sulfate, tungsten or tantalum to the polymer or polymer composite or the material selected may be radiopaque such as tantalum and gold to improve visibility of the implant. Further, plugs 122 may be included even if not needed as an insulator or heat sink, and may be used simply to seal ends of lumen 116 or as a radiopaque marker.

Alternatively, the ends of drug-eluting element 110 may be sealed by other methods. For example, methods described in provisional application No. 61/244,050, filed Sep. 20, 2009, incorporated by reference herein in its entirety, for sealing an end of a wire, may be used to seal ends of drug-eluting element 110. Further, as shown in FIG. 10, the ends of wall 114 may be swaged to close off the ends of drug-eluting element 110, as shown at 124. For example, a tube that is longer than drug eluting element 110 may be cut by a curved press that simultaneously presses the walls 114 at the location of the cut together, as shown in FIG. 10. This closes off the ends of lumen 116 and provides a surface to fuse or otherwise connect drug-eluting elements 110 to cylindrical elements 102. In another embodiment, shown in FIG. 11, the ends of drug-eluting element 110 are tapered such that the diameter at the ends is smaller than the diameter in the middle portion of drug-eluting element 110. Such tapered drug-eluting elements 110 may assist in improved crimping and reducing the crossing profile of the stent, particularly in the regions where the drug-eluting elements 110 are joined to the cylindrical elements 102.

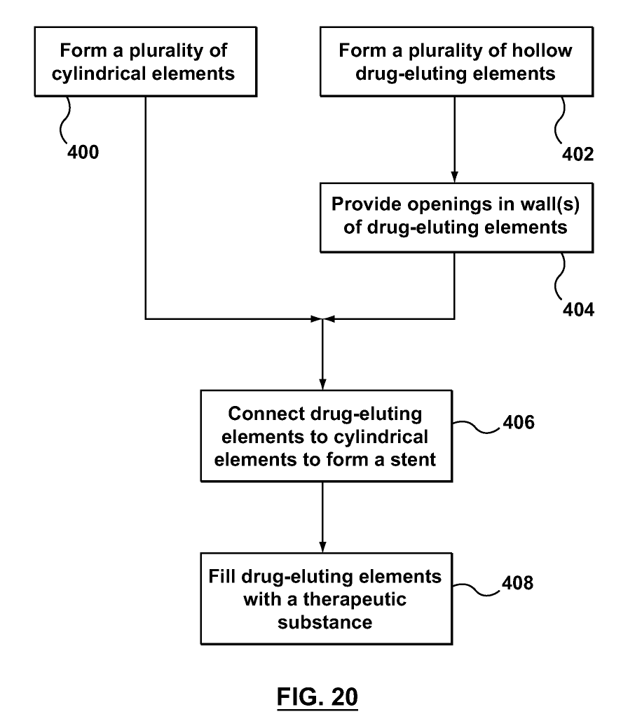

Another method for forming a stent 100 is outlined in FIG. 20. In step 400, cylindrical elements 102 are formed. In step 402, drug-eluting elements 110 are formed without a therapeutic substance disposed therein. In step 404, openings 112 are formed through the wall(s) 114 of drug-eluting elements 110. Openings 112 may be laser cut, drilled, etched, or otherwise provided through wall(s) 114 of drug-eluting element 110. Further, those of ordinary skill in the art would recognize that openings 112 may be formed prior to or after connecting drug-eluting elements 110 to cylindrical elements 102. In step 406, the drug-eluting elements 110 are connected to the cylindrical elements 102 to form a stent. After the drug-eluting elements 110 are connected to the cylindrical elements, lumens 116 of drug-eluting elements 110 are filled with a therapeutic substance 120 in step 408. Lumens 116 may be filled by any method known to those of ordinary skill in the art, for example, those listed above in the description of the method of FIG. 19. The method of FIG. 20 fills the lumens 116 of drug-eluting elements 110 after the drug-eluting elements have been connected to cylindrical elements 102, instead of before as in the method of FIG. 19.

Those of ordinary skill in the art would recognize that in some situations, some of the drug-eluting elements 110 could be filled with a therapeutic substance before being connected to cylindrical elements 102 and others could be filled with a therapeutic substance after being connected to cylindrical elements 102. For example, and not by way of limitation, if different therapeutic substances are used, drug-eluting elements to be filled with therapeutic substances that are sensitive to heat may be attached to the cylindrical elements prior to being filled, and drug-eluting elements to be filled with therapeutic substances that are not sensitive to heat may be filled prior to being attached to the cylindrical elements.

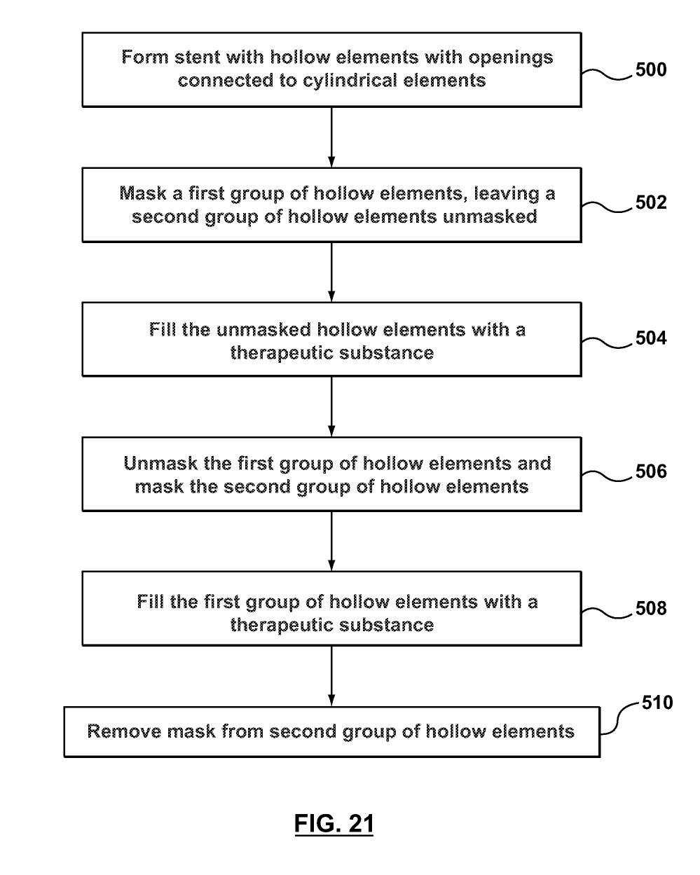

FIG. 21 outlines a method for filling a stent with drug-eluting elements 110 connected to cylindrical elements 102. In particular, the method of FIG. 21 may be utilized, for example, after step 406 of FIG. 20. Further, the method of claim 21 is particularly useful when some of the drug-eluting elements are to be filled with a first therapeutic substance and other of the drug-eluting elements are to be filled with a second therapeutic substance different from the first therapeutic substance. Thus, in step 500, a stent is formed with unfilled drug-eluting elements 110 connected between cylindrical elements 102. In step 502, a first group of unfilled drug-eluting elements are masked, leaving a second group of unfilled drug-eluting elements unmasked. In step 504, the stent is exposed to a first therapeutic substance to fill the unmasked second group of drug-eluting elements with the first therapeutic substance. The mask on the first group of drug-eluting elements prevents them from being filled. The drug-eluting elements may be filled utilizing any method known to those of ordinary skill in the art, including, but not limited to, the methods described above with respect to FIG. 19. In step 506, the mask is removed from first group of unfilled drug-eluting elements and the second group of drug-eluting elements (now filled) is masked. In step 508, the stent is exposed to a second therapeutic substance to fill the unmasked first group of drug-eluting elements with the second therapeutic substance. The mask on the second group of drug-eluting elements prevents them from being filled. The drug-eluting elements may be filled utilizing any method known to those of ordinary skill in the art, including, but not limited to, the methods described above with respect to FIG. 19. In step 510, the mask is removed from the second group of drug-eluting elements, leaving a stent with the first group of drug-eluting elements filled with the second therapeutic substance and the second group of drug-eluting elements filled with the first therapeutic substance. Those of ordinary skill in the art are familiar with suitable masks and methods of applying and removing such masks.

Stent 100 may be used conventionally in blood vessels of the body to support such a vessel after an angioplasty procedure. It is known that certain drugs eluted from stents may prevent restenosis or other complications associated with angioplasty or stents. Stent 100 may alternatively be used in other organs or tissues of the body for delivery of drugs to treat tumors, inflammation, erectile dysfunction, nervous conditions, or other conditions that would be apparent to those skilled in the art.

The therapeutic substance or drug 120 may include, but is not limited to, antineoplastic, antimitotic, antiinflammatory, antiplatelet, anticoagulant, antifibrin, antithrombin, antiproliferative, antibiotic, antioxidant, and antiallergic substances as well as combinations thereof. Examples of such antineoplastics and/or antimitotics include paclitaxel (e.g., TAXOL® by Bristol-Myers Squibb Co., Stamford, Conn.), docetaxel (e.g., Taxotere® from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include ABT-578 (a synthetic analog of rapamycin), rapamycin (sirolimus), zotarolimus, everolimus, angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include nitric oxide, alpha-interferon, genetically engineered epithelial cells, and dexamethasone. In other examples, the therapeutic substance is a radioactive isotope for implantable device usage in radiotherapeutic procedures. Examples of radioactive isotopes include, but are not limited to phosphorus (P³²), palladium (Pd¹⁰³), cesium (Cs¹³¹), Iridium (I¹⁹²) and iodine (I¹²⁵). While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions.

Further, a carrier may be used with the therapeutic substance or drug. Examples of suitable carriers include, but are not limited to, ethanol, acetone, tetrahydrofuran, dimethylsulfoxide, a combination thereof, or other suitable carriers known to those skilled in the art. Still further, a surfactant may be formulated with the drug and the solvent to aid elution of the drug.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. Furthermore, there is no intention to be bound by any expressed or implied theory presented in the preceding technical field, background, brief summary or the detailed description. All patents and publications discussed herein are incorporated by reference herein in their entirety.

What is claimed is:

1. A stent comprising:
a plurality of generally cylindrical elements aligned generally along a common longitudinal axis, wherein the plurality of cylindrical elements includes at least a first cylindrical element having a plurality of first struts connected together by a plurality of first crowns and a second cylindrical element adjacent to the first cylindrical element and having a plurality second struts connected together by a plurality of second crowns; and
a hollow element connected to the first cylindrical element and connected to the second cylindrical element, wherein the hollow element includes a therapeutic substance disposed within a lumen of the hollow element, and wherein the hollow element includes at least one opening through a wall of the hollow element.

2. The stent of claim 1, further comprising a plurality of hollow elements connected to the first cylindrical element and the second cylindrical element.

3. The stent of claim 2, wherein the opening of at least one of the plurality of hollow elements is disposed through an abluminal surface of the hollow element and the opening of at least another one of the plurality of hollow elements is disposed through a luminal surface of the hollow element.

4. The stent of claim 3, wherein the therapeutic substance in the lumen of the hollow elements with openings through the abluminal surface is different from the therapeutic substance of the hollow elements with openings through the luminal surface.

5. The stent of claim 1, further comprising a plurality of hollow elements connected between each of the plurality of cylindrical elements.

6. The stent of claim 1, wherein the hollow element is connected to a peak first crown of the first cylindrical element and a peak second crown of the second cylindrical element.

7. The stent of claim 1, wherein the hollow element is connected to a valley first crown of the first cylindrical element and a valley second crown of the second cylindrical element.

8. The stent of claim 1, wherein the hollow element is connected to one of the plurality of first struts of the first cylindrical element and to one of the plurality of second struts of the second cylindrical element.

9. The stent of claim 1, wherein the hollow element is fused to the first cylindrical element and to the second cylindrical element.

10. The stent of claim 1, wherein ends of the lumen of the hollow element are closed.

11. The stent of claim 10, wherein the ends are closed with a plug made from a radiopaque material.

12. The stent of claim 11, wherein the radiopaque material is tantalum.

13. The stent of claim 1, wherein at least one of the drug-eluting elements is curved.

14. A method of forming a stent comprising the steps of:
forming a plurality of cylindrical elements, wherein each cylindrical element includes a plurality of struts connected together by a series of crowns;
forming a plurality of hollow elements;
forming at least one opening through a wall of the hollow elements;
filling a lumen of the hollow elements with a therapeutic substance; and
connecting the hollow elements between the plurality of cylindrical elements such that ends of each hollow element are connected to adjacent cylindrical elements and such that the cylindrical elements are connected together and aligned generally along a common longitudinal axis to form the stent.

15. The method of claim 14, wherein the step of connecting the hollow elements between the cylindrical elements is completed prior to the step of filling the hollow elements with a therapeutic substance.

16. The method of claim 14, wherein the step of connecting the hollow elements between the cylindrical elements is completed after the step of filling the hollow elements with a therapeutic substance.

17. The method of claim 14, wherein the step of connecting the hollow elements to the cylindrical elements comprises fusing the hollow elements to the cylindrical elements.

18. The method of claim 14, wherein at least two of the hollow elements are filled with different therapeutic substances.

19. The method of claim 14, wherein when connected to the cylindrical elements, the opening of at least one of the hollow elements is disposed through a luminal wall of the hollow element and the opening of at least another one of the hollow elements is disposed through an abluminal wall of the hollow element.

20. The method of claim 19, wherein the therapeutic substance disposed in the hollow elements having the luminal wall opening is different from the therapeutic substance disposed in the hollow elements having the abluminal wall opening.
